(19) United States
(12) Patent Application Publication
(10) Pub. No.: US 2026/0263659 A1
(43) Pub. Date: Sep. 10, 2026

(54) METHOD FOR PRODUCING ADIPOSE TISSUE-DERIVED EXTRACELLULAR MATRIX (ECM) AND MEDICAL COMPOSITION COMPRISING SAME

(71) Applicant: CGBIO CO., LTD., Seoul (KR)

(72) Inventor: Gyumin KIM, Seoul (KR)

(21) Appl. No.: 19/471,613

(22) PCT Filed: Apr. 10, 2023
(86) PCT No.: PCT/KR2023/004786

§ 371 Date: Oct. 1, 2025

(51) Int. Cl. A61L 27/36; A61L 27/24

(52) U.S. Cl. CPC A61L 27/3633; A61L 27/24; A61L 27/3687; A61L 27/3691

(57) ABSTRACT

Provided is a method for producing an adipose tissue-derived extracellular matrix (ECM) in powder form. In an embodiment, the method for producing an adipose tissue-derived ECM may comprise: (i) a step of preparing a human-derived adipose tissue that does not undergo separate delipidation and decellularization processes; (ii) a delipidation step of removing lipid components from the adipose tissue; (iii) a decellularization step of removing cells and immunogens from the adipose tissue from which the lipid components have been removed; (iv) a washing step of washing the adipose tissue from which the cells and the immunogens have been removed; (v) a freeze-drying step of freeze-drying the washed adipose tissue; and (vi) a powdering step of powdering the freeze-dried product.

### CROSS-REFERENCE TO RELATED APPLICATION

This application is the National Stage filing under 35 U.S.C. 371 of International Application No. PCT/KR2023/004786, filed on Apr. 10, 2023.

### TECHNICAL FIELD

The present disclosure relates to a method for preparing an adipose tissue-derived extracellular matrix (ECM) and a medical composition including the same.

### BACKGROUND ART

The extracellular matrix (ECM) is a biocompatible polymer material containing collagen, fibronectin, and laminin, and is needed in regenerating injured tissues or organs. In the meantime, human body-derived ECM was primarily extracted from subcutaneous adipose tissue obtained through liposuction.

Meanwhile, adipose tissue is receiving increasing attention both domestically and internationally for its economic and industrial interests as well as its safety and effectiveness as a biomaterial. Adipose tissue is one of loose connective tissues composed of adipocytes, preadipocytes, fibroblasts, vascular endothelial cells, and various immune cells. Adipose tissue contains ECM such as collagen, elastin, laminin, fibronectin, glucosaminoglucan, etc. However, the adipose tissue is currently considered medical waste and is currently entirely incinerated.

Attempts are being made to utilize such an adipose tissue as a biomaterial.

For example, Renuva®, a human body adipose tissue-derived ECM product, has been reported to be effective in tissue repair when applied to defect sites such as foot ulcers. As another example, Allofill®, a human body adipose tissue-derived extracellular matrix product, has been reported to be effective in improving wrinkles. However, since medical products such as Renuva® and Allofill® are provided as a simple formulation of micronized human body adipose tissue-derived extracellular matrix powder hydrated in sterile saline solution, there is a disadvantage in that it is difficult for them to maintain volume in the body after use.

In addition, there have been attempts to use extracellular matrix powder derived from conventional adipose tissue as a medical composition, but there has been a problem in that tissue damage is caused by reagents used in the delipidation and/or decellularization process.

### DISCLOSURE

### Technical Problem

The present disclosure is intended to solve the above problems, and can allow the obtained adipose tissue-derived extracellular matrix to contain a high concentration of collagen while significantly improving the efficiency of delipidation and decellularization.

### Technical Solution

In order to solve the above problems, the present disclosure provides a method for preparing an adipose tissue-derived extracellular matrix (ECM) in powder form, the method including: step of preparing a human body-derived adipose tissue on which separate delipidation and decellularization processes have not been performed; (ii) a delipidation step of removing lipid components from the adipose tissue; (iii) a decellularization step of removing cells and immunogens from the adipose tissue from which the lipid components have been removed; (iv) a washing step of washing the adipose tissue from which the cells and immunogens have been removed; (v) a freeze-drying step of freeze-drying the washed adipose tissue; and (vi) a powdering step of powdering the freeze-dried lyophilized product.

### Advantageous Effects

According to one embodiment of the present disclosure, while improving the decellularization efficiency and, at the same time, including two or more delipidation processes in the overall process to significantly improve the delipidation efficiency, a high concentration of collagen can be allowed to be contained in the obtained adipose tissue-derived extracellular matrix.

### BRIEF DESCRIPTION OF DRAWINGS

FIG. 1 is a schematic diagram illustrating a method for preparing an adipose tissue-derived extracellular matrix in the powder form according to one embodiment of the present disclosure.

FIG. 2 illustrates a preparation step of human body-derived adipose tissue on which separate delipidation and decellularization processes have not been performed according to one embodiment of the present disclosure.

FIGS. 3, 4, and 5 illustrate a delipidation step of removing lipid components from the adipose tissue according to one embodiment of the present disclosure, wherein FIG. 3 illustrates that a human body-derived adipose tissue is washed, FIG. 4 illustrates that lipid components from the adipose tissue during the delipidation process are dissolved, and FIG. 5 illustrates that the adipose tissue is physically scraped to remove the lipid components during the delipidation process.

FIG. 6 illustrates a decellularization step of removing cells and immunogens from the adipose tissue from which the lipid components have been removed according to one embodiment of the present disclosure, and FIG. 7 illustrates that performance of the decellularization step has been completed according to FIG. 6.

FIG. 8 illustrates that the adipose tissue-derived extracellular matrix in the powder form has been obtained as a result of the powdering step of powdering the freeze-dried lyophilized product according to one embodiment of the present disclosure.

FIG. 9 illustrates that a photo obtained by photographing the stained adipose tissue-derived extracellular matrix with a microscope after staining an adipose tissue-derived extracellular matrix in the powder form with H&E, according to one embodiment of the present disclosure.

FIG. 10 illustrates a result of evaluating collagen content in the adipose tissue-derived extracellular matrix according to one embodiment of the present disclosure through a hydrosyproline assay.

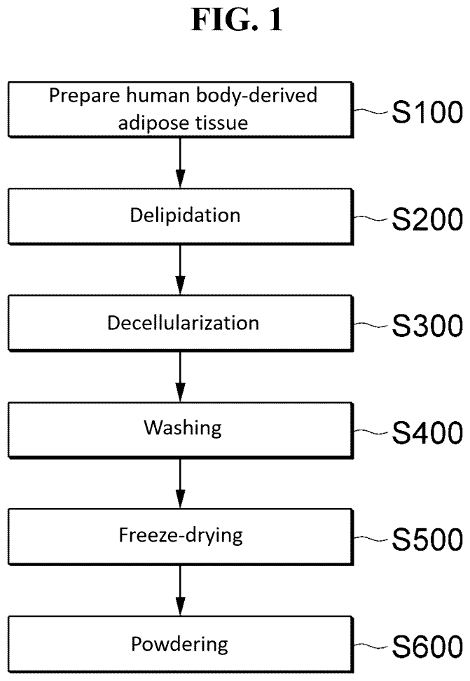

### MODE FOR INVENTION

The present disclosure provides a method for preparing an adipose tissue-derived extracellular matrix, which allows a high concentration of collagen to be contained in the obtained adipose tissue-derived extracellular matrix while significantly improving the delipidation efficiency and decellularization efficiency, compared to conventionally developed methods of obtaining an extracellular matrix from adipose tissue.

Therefore, in one aspect, the present disclosure relates to a method for preparing an adipose tissue-derived extracellular matrix, which includes two or more delipidation steps and can perform delipidation and decellularization at the same time, including:
(i) a step of preparing a human body-derived adipose tissue on which separate delipidation and decellularization processes have not been performed (S100);
(ii) a delipidation step of removing lipid components from the adipose tissue (S200);
(iii) a decellularization step of removing cells and immunogens from the adipose tissue from which the lipid components have been removed (S300);
(iv) a washing step of washing the adipose tissue from which the cells and immunogens have been removed (S400);
(v) a freeze-drying step of freeze-drying the washed adipose tissue (S500); and
(vi) a powdering step of powdering the freeze-dried lyophilizate (S600).

First, (i) a step of preparing a human body-derived adipose tissue on which separate delipidation and decellularization processes have not been performed (S100) will be described.

In the present disclosure, the adipose tissue-derived extracellular matrix may include one in the powder form. For example, the powder form may have an average particle diameter of 50 μm to 900 μm, for example, 400 μm to 600 μm.

In the present disclosure, the adipose tissue-derived extracellular matrix may be used as a medical material, and for example, it may promote autologous fat production and induce autologous tissue organization after in vivo transplantation.

In one embodiment, the medical material may be used as a tissue repair agent, filler, anti-adhesion agent, cosmetic aid, arthritis treatment agent, wound dressing agent, hemostatic agent, or lipodystrophy treatment agent.

In one embodiment, the adipose tissue-derived extracellular matrix may be prepared in at least one of a sheet type form, a hydrogel type form, and an injectable type form. For example, the adipose tissue-derived extracellular matrix may be prepared as a sheet type wound dressing agent. For example, the adipose tissue-derived extracellular matrix may be prepared as a hydrogel type cosmetic aid. For example, the adipose tissue-derived extracellular matrix may be prepared as an injectable type filler.

In one embodiment, the extracellular matrix (ECM) refers to a complex aggregate of biopolymers that fills the inside of a tissue or extracellular spaces. The components of the ECM may vary depending on the type of the cell or the differentiation degree of the cell, and may be composed of fibrous proteins such as collagen, elastin, etc., complex proteins such as proteoglycans, glycosaminoglycans, etc., and cell-adhesive glycoproteins such as fibronectin, laminin, etc.

In one embodiment, the adipose tissue may be an adipose tissue of homologous or xenogeneic origin. The “homogeneous” refers to humans, and “xenogeneic” may refer to animals other than humans, i.e., fishes, etc. as well as mammals such as pigs, cows, horses, etc.

In one embodiment, the adipose tissue may be a human body-derived adipose tissue that has not undergone separate delipidation and decellularization processes.

In one embodiment, the adipose tissue may be derived from lung fat of the human body.

The lung fat-derived adipose tissue applied to the present disclosure has confirmed effectiveness in current tissue repair and has demonstrated biological stability, and since the lung fat-derived adipose tissue contains collagen, elastin, etc., there is an advantage in that it may be used as a raw material for artificial skin, pharmaceuticals, and medical devices.

In addition, the extracellular matrix (ECM) derived from human body lung fat-derived adipose tissue applied to the present disclosure has excellent biocompatibility and tissue regenerative power, thereby having great therapeutic effects.

Therefore, the present disclosure can prepare artificial skin with excellent biocompatibility and tissue regenerative power and manufacture pharmaceuticals and medical devices with great therapeutic effects by preparing an extracellular matrix (ECM) derived from the human body lung fat-derived adipose tissue in powder form.

Next, (ii) the delipidation step (S200) of removing lipid components from the adipose tissue will be described.

In one embodiment, the delipidation step refers to removing lipid components from the adipose tissue, and for example, the delipidation step may include washing the adipose tissue and then removing the lipid components using an organic solvent.

For example, washing the adipose tissue may be performed once to ten times by introducing the adipose tissue into a washing bath containing PBS 3 to 6 times the weight of the adipose tissue at 4 to 45° C. conditions overnight and/or for 5 to 60 minutes each.

For example, removing lipid components using the organic solvent may be performed using any one of a stirrer, a shaking water bath, and a shaking incubator, and for example, it may be performed in the shaking incubator at 120 rpm.

For example, the organic solvent may include a polar solvent, a non-polar solvent, or a mixed solvent thereof. Water, an alcohol, or a mixed solution thereof may be used as the polar solvent, and methanol, ethanol, or isopropyl alcohol may be used as the alcohol. Hexane, heptane, octane, or a mixed solution thereof may be used as the non-polar solvent. For example, as the organic solvent, a high-concentration alcohol may be used, specifically 70% isopropyl alcohol may be used.

The present disclosure can primarily remove lipid components using an organic solvent as described above, and then physically scrape the adipose tissue to secondarily remove any remaining lipid components.

For example, the adipose tissue can be physically scraped using forceps or a spatula to secondarily remove the lipid components.

Next, (iii) the decellularization step ($300) of removing cells and immunogens from the adipose tissue from which the lipid components have been removed is described. In one embodiment, decellularization refers to the removal of cellular components other than the extracellular matrix, for example, the nucleus, cell membrane, nucleic acids, etc., from the tissue.

When conventional solutions, such as sodium hydroxide, have been used, there have been problems in that delipidation and decellularization are not sufficiently achieved during short-term treatment, and denature of residual proteins within the scaffold is caused, the microstructure of the extracellular matrix is collapsed, and growth factors are excessively removed during long-term treatment, and in order to solve these problems, the decellularization step of the present disclosure is characterized by treatment with a hypotonic solution containing an ionic surfactant or a non-ionic surfactant. The ionic surfactant may be preferably applied. Furthermore, the present disclosure greatly enhances the delipidation effect by removing lipid components three times during the decellularization step.

In one embodiment, the ionic surfactant may be one or more selected from the group consisting of a glutamate-based surfactant such as alkylbenzene sulfonate, alkyl sulfate, alkyl ether sulfate, alkoxylated amide, olefin sulfonate, alkyl xylene sulfonate, dialkyl sulfosuccinate, fatty acid ester sulfonate, alcohol sulfate, glycerol fatty acid ester, or the like, an isethionate-based surfactant, a phosphate-based surfactant such as lauryl phosphate, laureth-1-phosphate, or the like, a taurate-based surfactant, an alkoxylated alcohol-based surfactant, an alkyl carboxylate-based surfactant, hydroxyalkyl, quaternary ammonium salt, and ethoxylated alkyl, but the present disclosure is not limited thereto.

Preferably, the ionic surfactant may be one or more selected from the group consisting of sodium dodecyl sulfate (SDS), sodium laureth sulfate (SLS), sodium lauryl ether sulfate (SLES), sodium myreth sulfate (SMES), dioctyl sodium sulfosuccinate (DSS), perfluorooctanesulfonate (PFOS), perfluorobutanesulfonate (PFBS), perfluorononanoate (PFNA), perfluorooctanoate (PFOA), sodium stearate, sodium lauroyl sarcosinate, cetrimonium bromide (CTAB), cetylpyridinium chloride (CPC), benzalkonium chloride (BAC), benzethonium chloride (BZT), dimethyldioctadecylammonium chloride (DDAC), and dioctadecyldimethylammonium bromide (DODAB), and more preferably, the ionic surfactant may be sodium dodecyl sulfate (SDS), but the present disclosure is not limited thereto.

In the present disclosure, an ionic surfactant which enables delipidation and causes less damage to tissue while having excellent cell removal efficiency, preferably an alkyl sulfate surfactant, and more preferably sodium dodecylsulfate (SDS), may be applied.

The concentration of sodium dodecylsulfate (SDS) is within a range enabling a high collagen content by enabling delipidation and causing less damage to tissue while having excellent decellularization efficiency from an adipose tissue-derived extracellular matrix, and may be 0.1 to 0.8% (w/v), preferably 0.45 to 0.65% (w/v), in a hypotonic solution.

Since the term “hypotonic” used in the present disclosure is a state in which, when comparing the osmotic pressures of two solutions with a semipermeable membrane being interposed between the two solutions, the inside has a higher osmotic pressure than the outside, i.e., a state in which the concentration inside the membrane is higher than the concentration outside the membrane, it refers to a state in which a net movement of water is towards the inside of the membrane, and the rate of osmosis phenomenon, which is such a passive diffusion phenomenon of water, increases or decreases in proportion to the concentration gradient between the two solutions, and when the concentration difference disappears, the net movement of water disappears.

In the present disclosure, a “hypotonic solution” refers to a solution with an osmolarity of approximately 270 mOsm/L or less, the hypotonic solution is used in treatments for affecting cell lysis, the hypotonic solution flows into cells through the cell membrane via osmosis, and at this time, the cells of the tissue may be caused to expand and rupture, thereby eliminating any residual cells.

In addition, in the present disclosure, the term “osmolarity” is an expression of the osmotic concentration per volume of solution (mOsm/L), and the osmotic concentrations of plasma in the body and other body fluids are 270 to 300 mOsm/L.

At this time, if the osmolarity of the solution is about 270 mOsm/L or less, it is defined as a hypotonic solution, if the osmolarity of the solution is about 270 to 300 mOsm/L, it is defined as an isotonic solution, and if the osmolarity of the solution is about 300 mOsm/L or more, it is defined as a hypertonic solution.

The hypotonic solution of the present disclosure is characterized by having an osmolarity of 270 mOsm/L or less. It preferably contains 5 to 20 mM of Tris-HCl, 0.1 to 1.5% of ethylenediaminetetraacetic acid (EDTA), and 0.01 to 0.05 M of sodium hydroxide (NaOH), and more preferably 8 to 12 mM of Tris-HCl, 0.7 to 1.2% (w/v) of ethylenediaminetetraacetic acid (EDTA), and 0.01 to 0.02 M of sodium hydroxide (NaOH). The hypotonic solution may contain the ionic surfactant.

In addition, in the present disclosure, the hypotonic solution can be treated to adipose tissue for 2 to 10 hours, preferably 3 to 9 hours, and more preferably 4 to 8 hours, but the present disclosure is not limited thereto.

In this way, the present disclosure enables the tissue to have a high collagen content since tissue damage is minimized while increasing decellularization and delipidation efficiencies by tertiarily removing the lipid components along with a decellularization process in which adipose tissue, from which lipid components have been secondarily removed, is immersed in a hypotonic solution containing 5 to 20 mM of Tris-HCl, 0.1 to 1.5% of ethylenediaminetetraacetic acid (EDTA), 0.01 to 0.05 M of sodium hydroxide (NaOH), and 0.45 to 0.65% of SDS to remove cells and immunogens.

In another embodiment, the present disclosure may further include performing sonication during the decellularization step. For example, the decellularization effect can be increased through sonication after performing the decellularization process of immersing the adipose tissue in the hypotonic solution described above.

Next, (iv) the washing step (S400) of washing the adipose tissue from which the cells and immunogens have been removed will be described.

In one embodiment, the washing step may be performed one time or more.

For example, the buffer solution may include one or more selected from the group consisting of phosphate buffer saline (PBS), distilled water, normal saline, Hank's balanced salt solution (HBSS), Tris buffered saline (TBS), N-Tris(hydroxymethyl)methyl-3-aminopropanesulfonic acid (TAPS) buffer solution, N,N-Bis(2-hydroxyethyl)glycine (Bicine) buffer solution, 4-(2-hydroxyethyl)-1-piperazineethanesulfonic acid (HEPES) buffer solution, N-Tris(hydroxymethyl)methyl-2-aminoethanesulfonicd acid (TES) buffer solution, piperazine-N,N′-bis(2-ethanesulfonic acid (PIPES) buffer solution, cacodylate buffer solution, 2-(N-morpholino) ethanesulfonic acid (MES) buffer solution, minimum essential media (MEM), Dulbecco's modified eagle media (DMEM), RPMI1640, Iscove's modified Dulbecco's media (IMDM), defined Keratinocyte-SFM (without bovine pituitary extract (BPE)), Keratinocyte-SFM (with BPE), KnockOut D-MEM, AmnioMAX-II complete medium, AmnioMAX-C100 complete medium, and mixtures thereof.

In one embodiment, washing using the buffer may be performed overnight and/or for 5 to 60 minutes each once to ten times at temperature conditions of 4 to 45° C., preferably overnight and/or for 5 to 30 minutes each once to five times.

Next, (v) the freeze-drying step (S500) of freeze-drying the washed adipose tissue will be described.

The freeze-drying step involves freeze-drying the washed adipose tissue, and for example, a method in which the tissue is rapidly cooled in a frozen state and then moisture is absorbed by vacuum may be applied, and may enable powdering to be easily performed.

For example, freeze-drying may be performed at −90° C. to −30° C. for 12 to 84 hours.

Next, (vi) the powdering step (S600) of powdering the freeze-dried lyophilized product will be described.

In the powdering step, the lyophilized product may be an extracellular matrix that has completed delipidation and decellularization.

In one embodiment, the powdered extracellular matrix powder may have an average particle diameter of 50 μm to 900 μm, e.g., 400 μm to 600 μm.

Hereinafter, the present disclosure will be described in more detail through Examples. However, Examples are intended to illustrate the present disclosure and the scope of the present disclosure is not limited to these Examples.

### Example 1: Preparation of Adipose Tissue-Derived Extracellular Matrix

A human body lung peripulmonary adipose tissue on which separate delipidation or decellularization processes have not performed was obtained.

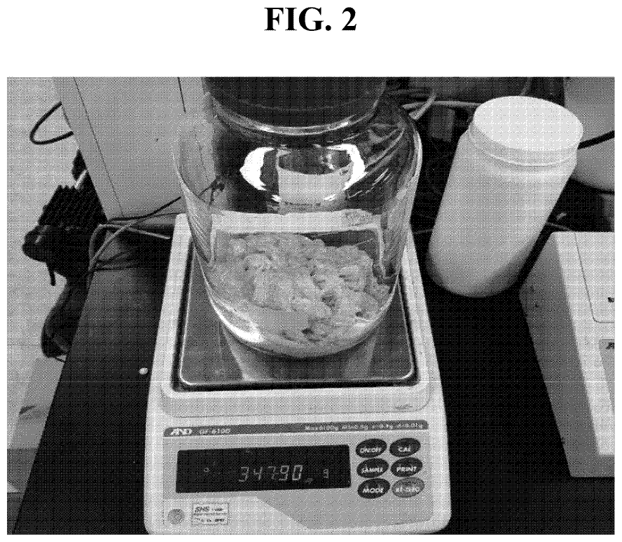

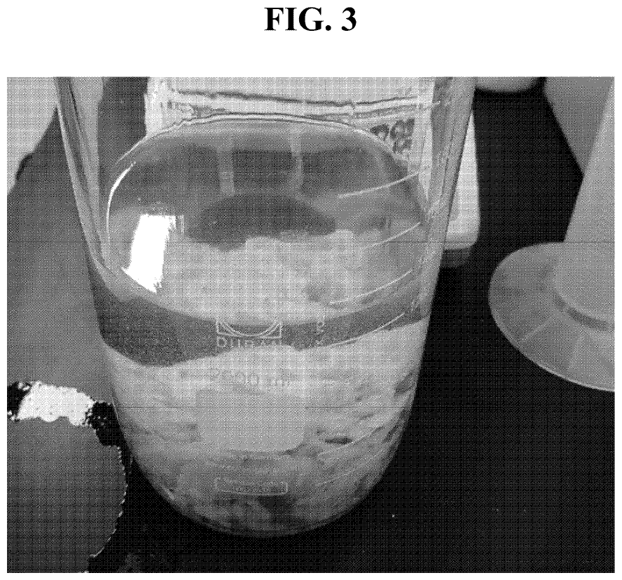

As shown in FIG. 2, the adipose tissue was introduced into a washing bath containing PBS 5 times the weight of the adipose tissue and PBS washing was performed overnight at 5° C.

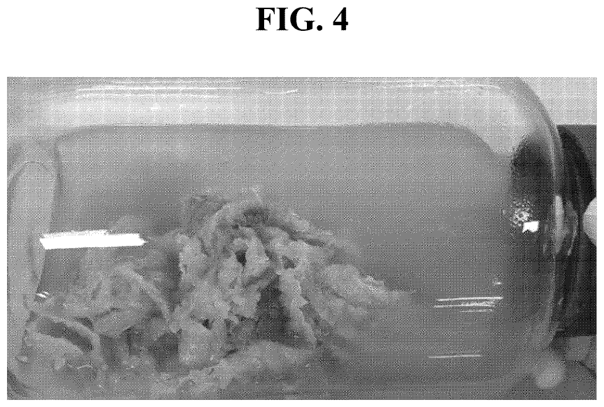

The washed adipose tissue shown in FIG. 3 was introduced into a shaking incubator containing 70% isopropyl alcohol (IPA) and the lipid components were primarily removed at 120 rpm. As a result, it can be confirmed that the lipid components were dissolved in the organic solvent as shown in FIG. 4.

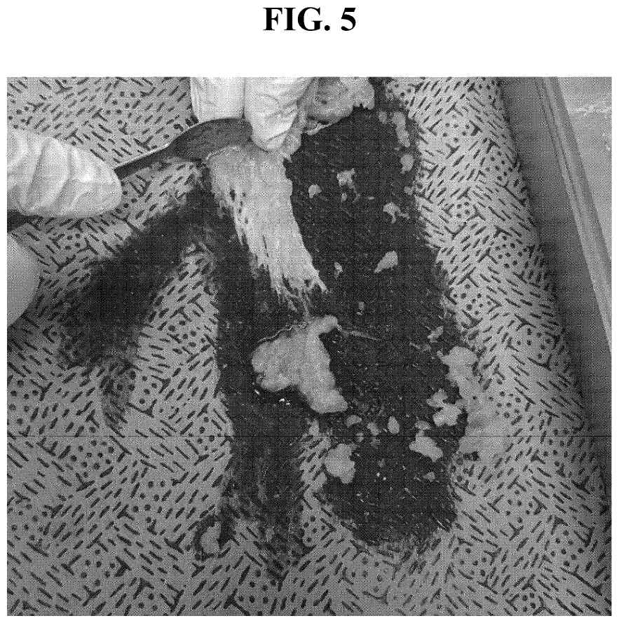

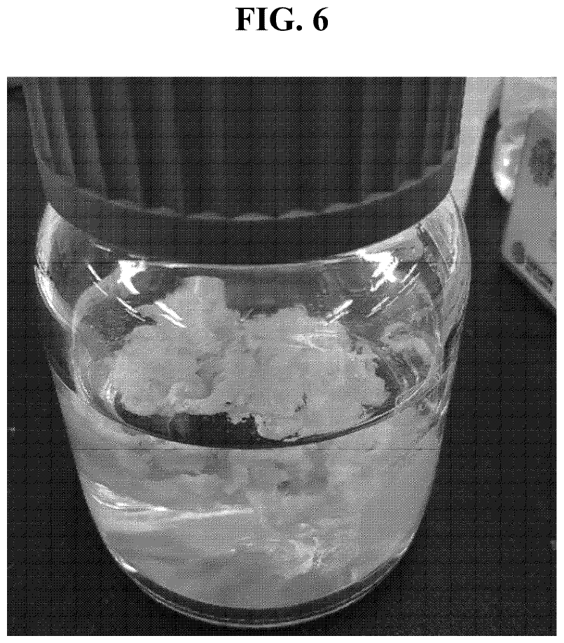

After the lipid components were primarily removed with the organic solvent, the adipose tissue was physically scraped using forceps or a spatula to secondarily remove the lipid components as shown in FIG. 5, and the tissue was washed five times with aseptic water.

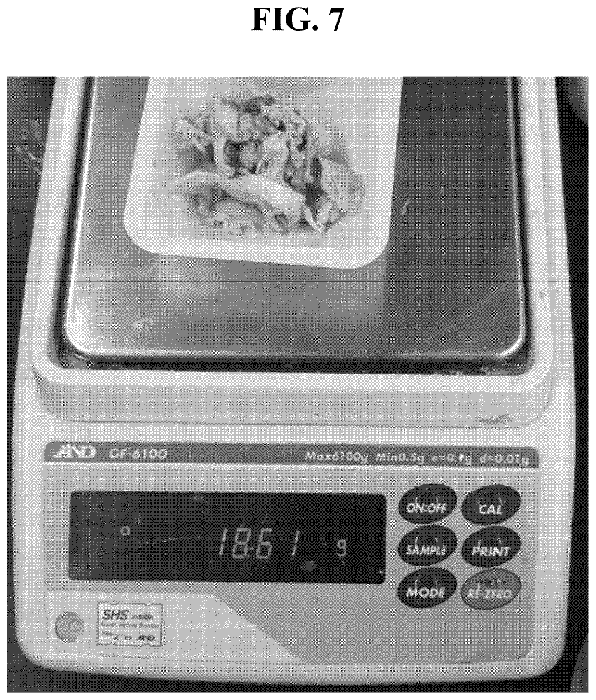

The adipose tissue from which the lipid components were secondarily removed was immersed in a hypotonic solution containing 10 mM of Tris-HCl, 1 g of EDTA, 13.5 mL of 1 M NaOH, and 0.5% SDS as shown in FIG. 6, and then reacted for 5 hours to tertiarily remove the lipid components along with the decellularization process of removing cells and immunogens.

Afterwards, in order to remove lipid components, cells, and SDS remaining on the surface, the adipose tissue was introduced into a shaking incubator containing 5° C. PBS, and PBS washing was performed. As a result, an adipose tissue from which the lipid components and cellular components were removed was obtained as shown in FIG. 7.

The adipose tissue from which the lipid components and cellular components were removed was freeze-dried at −60° C. for 48 hours using a freeze-dryer.

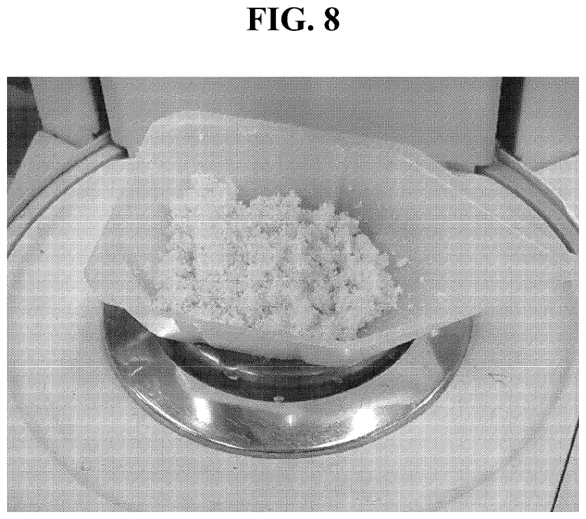

The freeze-dried lyophilized product was ground using a blender to obtain an adipose tissue-derived extracellular matrix powder as shown in FIG. 8.

### Example 2: Delipidation and Decellularization Evaluation (H&E Staining)

The delipidation and decellularization evaluation was performed on the adipose tissue-derived extracellular matrix obtained through Example 1 via histological examination using H&E staining.

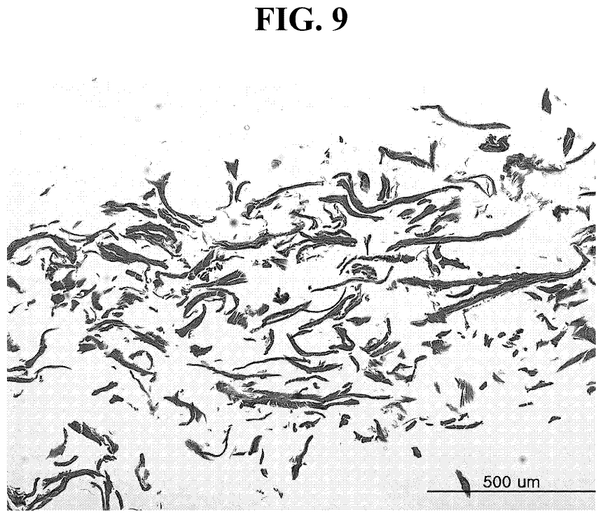

A paraffin block was sectioned to a 4 μm thickness and then dried to fabricate paraffin sections. Afterwards, for the deparaffinization process, the paraffin sections were reacted in xylene 3 times for 5 minutes each, 100% ethanol 3 times for 2 minutes each, 90% ethanol once for 1 minute each, 80% ethanol once for 1 minute each, and 70% ethanol once for 1 minute each, and then washed in running water for 10 minutes. After reacting the washed reaction products in a hematoxylin staining solution for 10 minutes, the reaction products were washed in running water for 3 minutes, reacted in an eosin staining solution for 10 minutes, and then washed in running water until the eosin staining solution did not appear. After reacting the washed reaction products in 70% ethanol 10 times for 1 second each, 80% ethanol 10 times for 1 second each, 90% ethanol 10 times for 1 second each, 100% ethanol 2 times for 1 minute each, and xylene 3 times for 3 minutes each, they were mounted with a mounting solution, and the adipose tissue-derived extracellular matrix powder of Example 1 was photographed with an optical microscope (Olympus BX51, H&E staining) and a scanning electron microscope (Hitachi S-4700, Japan), and is shown in FIG. 9.

As shown in FIG. 9, as a result of performing H&E staining on the adipose tissue treated with a hypotonic solution containing 0.5% SDS and photographing the H&E staining-performed adipose tissue with a microscope, lipid components and cellular components were not observed.

Meanwhile, as results of comparing the delipidation and decellularization effects of a solution of 1% Triton X-100, 1% Tween 80, and 0.1% SDS, a hypotonic solution containing 0.25% SDS, and a hypotonic solution containing 0.5% SDS under the same treatment conditions for the same time, the adipose tissue treated with the solution of 1% Triton X-100, 1% Tween 80, and 0.1% SDS retained lipid components and cellular components, and the adipose tissue treated with the hypotonic solution containing 0.25% SDS retained some lipid components, although no cellular components were observed (data not shown). In contrast, neither lipid components nor cellular components were observed from the adipose tissue treated with the hypotonic solution containing 0.5% SDS.

In addition, in the adipose tissue that was sonicated for 1 hour and the group that was sonicated for 1 hour and treated with 1% Triton X-100, it could be observed that the cells were not removed and the tissue was damaged.

In this way, treatment with the hypotonic solution containing 0.5% SDS not only effectively removed cellular components but also removed some residual lipid components, finally confirming that the present disclosure has the effect of removing lipid components in three stages.

### Example 3: Collagen Content Measurement

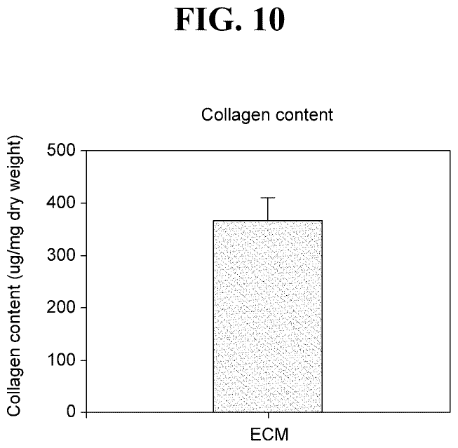

After 10 mg of the adipose tissue-derived extracellular matrix powder of the present disclosure and the control group (collagen fiber) were homogenized in 100 μL of distilled water, 100 μL of HCl (37% or ~12 M) was added, and hydrolysis was performed at 120° C. for 3 hours. After completing hydrolysis, the supernatant was obtained by performing centrifugation at a rate of 10,000 g for 3 minutes. The amount of hydroxyproline in the supernatant was measured at 560 nm absorbance using a hydroxyproline assay kit (MAK008-1KT, Sigma-Aldrich) to check the amount of collagen, which is shown in FIG. 10.

As shown in FIG. 10, the adipose tissue-derived extracellular matrix powder prepared according to an embodiment of the present disclosure was confirmed to contain a collagen content of 300 μg/mg or more, and even 350 μg/mg or more.

Hereinabove, since specific parts of the present disclosure content have been described in detail, it will be apparent to those skilled in the art that these specific descriptions are merely preferred embodiments and the scope of the present disclosure is not limited thereby. Therefore, the substantial scope of the present disclosure will be defined by the appended claims and equivalents thereof.

1. A method for preparing an adipose tissue-derived extracellular matrix (ECM) in powder form, the method comprising:
(i) a step of preparing a human body-derived adipose tissue on which separate delipidation and decellularization processes have not been performed;
(ii) a delipidation step of removing lipid components from the adipose tissue;
(iii) a decellularization step of removing cells and immunogens from the adipose tissue from which the lipid components have been removed;
(iv) a washing step of washing the adipose tissue from which the cells and immunogens have been removed;
(v) a freeze-drying step of freeze-drying the washed adipose tissue; and
(vi) a powdering step of powdering the freeze-dried lyophilized product.

2. The method of claim 1, wherein the delipidation step comprises washing the adipose tissue and then removing the lipid components using an organic solvent.

3. The method of claim 2, wherein the delipidation step further comprises removing the lipid components using the organic solvent, and then physically scraping the adipose tissue to secondarily remove the remaining lipid components.

4. The method of claim 1, wherein the decellularization step is treating with a hypotonic solution containing an ionic surfactant or a non-ionic surfactant, wherein the lipid components in the decellularization step are tertiarily removed.

5. The method of claim 4, wherein the hypotonic solution has an osmolarity of 270 mOsm/L or less.

6. The method of claim 1, wherein the washing step uses a buffer solution, the buffer solution includes one or more selected from the group consisting of phosphate buffer saline (PBS), distilled water, normal saline, Hank's balanced salt solution (HBSS), Tris buffered saline (TBS), N-Tris(hydroxymethyl)methyl-3-aminopropanesulfonic acid (TAPS) buffer solution, N,N-Bis(2-hydroxyethyl)glycine (Bicine) buffer solution, 4-(2-hydroxyethyl)-1-piperazineethanesulfonic acid (HEPES) buffer solution, N-Tris(hydroxymethyl)methyl-2-aminoethanesulfonicd acid (TES) buffer solution, piperazine-N,N′-bis(2-ethanesulfonic acid (PIPES) buffer solution, cacodylate buffer solution, 2-(N-morpholino) ethanesulfonic acid (MES) buffer solution, minimum essential media (MEM), Dulbecco's modified eagle media (DMEM), RPMI1640, Iscove's modified Dulbecco's media (IMDM), defined Keratinocyte-SFM (without bovine pituitary extract (BPE)), Keratinocyte-SFM (with BPE), KnockOut D-MEM, AmnioMAX-II complete medium, AmnioMAX-C100 complete medium, and mixtures thereof.

7. A medical composition in powder form comprising the human body-derived acellular dermal matrix prepared according to claim 1.

8. The medical composition in powder form of claim 7, wherein the medical composition in powder form is prepared in any one of a sheet type form, a hydrogel type form, and an injectable type form.
